(19) United States
(12) Patent Application Publication
(10) Pub. No.: US 2021/0202050 A1
(43) Pub. Date: Jul. 1, 2021

(54) METHOD FOR CALCULATING THICKNESS OF OXIDE FILM OF MARTENSITE HEAT-RESISTANT STEEL UNDER SUPERCRITICAL HIGH-TEMPERATURE STEAM

(71) Applicants: China Energy Science and Technology Research Institute Co., Ltd., Nanjing (CN); WUHAN UNIVERSITY, Wuhan (CN)

(72) Inventors: Yalin ZHANG, WUHAN (CN); Xue WANG, WUHAN (CN); Kai ZHANG, WUHAN (CN); Dejun REN, WUHAN (CN); Zhixiong ZUO, WUHAN (CN); Shengli LIU, WUHAN (CN); Weiping DING, WUHAN (CN); Qiaosheng HUANG, WUHAN (CN)

(21) Appl. No.: 17/136,155

(22) Filed: Dec. 29, 2020

(30) Foreign Application Priority Data
Dec. 30, 2019 (CN) 201911396580.X

(51) Int. Cl. G16C 60/00; G16C 20/10; G01B 21/08

(52) U.S. Cl. CPC G16C 60/00; G01B 21/085; G16C 20/10

(57) ABSTRACT

A method for calculating a thickness of an oxide film of a martensite heat-resistant steel under supercritical high-temperature steam is disclosed, which includes following steps: the martensite heat-resistant steel is a 9% Cr martensite heat-resistant steel; and a formula for calculating the thickness of the oxide film is

$$X = A exp ( \frac{- Q}{R T} ) {t}^{n} ,$$

which X is the thickness of the oxide film (μm), A is a constant coefficient, Q is an activation energy (J·mol⁻¹), R is a gas constant, T is temperature (° C.), and t is time (h).

### CROSS REFERENCES TO RELATED APPLICATIONS

The present application claims foreign priority of Chinese Patent Application No. 201911396580.X, filed on Dec. 30, 2019 in the State Intellectual Property Office of China, the disclosures of all of which are hereby incorporated by reference.

### TECHNICAL FIELD

The present disclosure relates to a method for calculating the thickness of an oxide film of martensite heat-resistant steel under supercritical high-temperature steam, in particular to a method for calculating the thickness of an oxide film of 9% Cr martensite heat-resistant steel under supercritical high-temperature steam environment.

### BACKGROUND ART

The 9% Cr martensitic heat-resistant steel mainly includes T/P91, T/P92, E911 and G115 (9Cr3W3Co) and other martensitic heat-resistant steels, and is widely used in high-temperature parts such as a main steam tube, a header, a superheater and a reheater of an ultra-supercritical boiler. In order to improve heat efficiency, reduce coal consumption and emission, the steam pressure and the temperature of the thermal power generating unit are continuously improved, and the problem of high-temperature oxidation of key components of the thermal power generating unit is also serious. The explosion to leakage, damage accidents and unit outages of the heated surface tube caused by oxidation corrosion of the high-temperature steam are detrimental to safe operation and economic benefit of the power plant.

In recent years, with broad attention paid to the problem of steam oxidation of high-temperature parts of thermal power generating units, the hazard of steam oxidation is widely known to the prior arts, and the research investment on steam oxidation is gradually increased at home and abroad. The main concern on engineering concept is a thickness of oxide-scale formed by the material in the high temperature steam. With the temperature increasing, the oxide film grows faster while the oxide-scale formed in a certain time is thicker, which leads to the following problems: firstly, the actual tube wall thickness decreases due to the thickening of the oxide film, so that the pressure borne by the tube wall is increased, even the tube wall is damaged by creep deformation; secondly, a thermal conductivity of the oxide film is low, so that temperature of the tube wall is increased, thus oxidation corrosion and failure are further accelerated; thirdly, when the oxide-scale reaches a certain thickness or is heated unevenly due to over temperature or frequent start/stop of the tubeline, part of the oxide-scale is peeled off under an action of stress, and the peeled oxide material may block the tubeline or enter a steam turbine to cause erosion of blades of the steam turbine and so on. Therefore, to calculate the thickness of the oxide-scale of the heat-resistant steel tubeline based on the working temperature and time is significant for judging the oxidation corrosion degree of the tubes and calculating residual service life, and eventually ensuring safe operation of the power plant.

An oxidation kinetic model of 9 Cr % heat-resistant steel in a high-temperature steam environment is used for calculating the thickness of the oxide scale. At present, in China and abroad, the research on the high-temperature steam oxidation kinetics of 9 Cr % heat-resistant steel mainly adopts a method of oxidation-weight-increment, and the research on the thickness of the oxide-scale is limited to an influence of a single variable quantity (such as the temperature of the steam or time). The running temperatures of different units are often different, the oxide-scale growth rates at different temperatures are also different, an oxidation kinetic model obtained under a single working condition cannot be suitable for other temperature conditions, which is not suitable for calculating the thickness of an oxide film in practice. The common methods for industrially measuring the thickness of the oxide-scale include scale washing method, sampling electron microscope measurement, micro-area analysis method, an ultrasonic detection method, etc. But the aforementioned methods have defects of such as high cost, long period, unstable precision, complex operation, need for tube cutting and so on.

### SUMMARY

The present application aims to solve problems in the prior art and provides a method for quickly calculating the thickness of the oxide film of the 9% Cr martensite heat-resistant steel tube, based on a high-temperature steam oxidation kinetic model of 9% Cr martensite heat-resistant steel and related experimental data, under the condition of known working temperature and time of the 9% Cr martensite heat-resistant steel tube.

In order to achieve the above purpose, the technical scheme disclosed by the present disclosure is as follows: a method for calculating a thickness of an oxide film of a martensite heat-resistant steel under supercritical high-temperature steam including that the martensite heat-resistant steel is 9% Cr martensite heat-resistant steel, and a formula for calculating the thickness of the oxide film under high-temperature steam is as follows:

$$X = A exp ( \frac{- Q}{R T} ) {t}^{n}$$

In which, X is the thickness of the oxide film, A is a constant coefficient, Q is an activation energy, R is a gas constant, T is temperature, and t is time.

The technical scheme is further designed as follows: the temperature of the high-temperature steam is in a range of 550-700° C., the steam pressure is in a range of 23.0-35.0 MPa, and the time is in a range of 200-20000 h.

In which, n in the formula is 0.5.

A mathematical relationship between the activation energy Q and the time t is: Q=39659.32t^{0.0092}.

The constant coefficient A is 11616.83.

According to the formula of calculating the thickness, when the oxidation time is 200 h, Q is 41628.07.

According to the formula of calculating the thickness, when the oxidation time is 600 h, Q is 42057.09.

According to the formula of calculating the thickness, when the oxidation time is 1000 h, Q is 42256.60.

According to the formula of calculating the thickness, when the oxidation time is 1500 h, the value of Q is 42414.76.

According to the formula of calculating the thickness, when the oxidation time is 2000 h, Q is 43426.66.

The beneficial effects of the present application are described hereinafter:

Provided that the 9% Cr martensitic heat-resistant steel is disposed in a high-temperature steam environment of 23.0-35.0 MPa and 550-700° C., running for 200-2000 h, a method for calculating the thickness of an oxide film generated therein is disclosed. The method breaks through a limitation that a common oxidation kinetics formula only can reflect the influence of time on the thickness of the oxide film, but simultaneously considers two factors of the steam temperature and the running time which have a largest influence on the thickness of the oxide film, and mathematically corrects the formula by combining actual data on the basis of the traditional parabolic rate model. The thickness of the oxidation film of the 9% Cr martensitic heat-resistant steel can be conveniently and quickly calculated according to the running time and the temperature without cutting a tube for measurement, so that the cost is saved, and the thickness of the oxidation film of the tube can be calculated without influencing normal running. In addition, the thickness of the oxide film of the 9% Cr martensite heat-resistant steel is calculated by the formula, the oxidation corrosion degree on inside of the tube wall can be reflected, also provided as references for calculating the residual service life of the components to make sure safe operation of a power plant unit.

### DETAILED DESCRIPTION

The metal oxidation kinetic model applied in the present disclosure includes following steps: linear velocity law, parabolic velocity law, logarithmic velocity law, and cubic velocity law. And the parabolic law may be applied when a ratio of a volume of the oxide to that of the metal is close to 1 but not more than 15%, the oxide layer may cover the metallic surface compactly meanwhile not crack due to excessive internal stress. Further, oxidation reaction is performed by mass diffusion within the oxide layer, namely, metallic cations diffuse outwards and oxygen anions diffuse inward, or cations and anions bi-directional diffuse, then new oxides are formed within the oxide layer. When an interface-reaction speed is faster than a diffusion speed, an oxide generation speed depends on the speed of the mass diffusion. The oxide film of 9% Cr heat-resistant steel is mainly divided into an inner layer and an outer layer, and growth of the oxide film disposed on the inner layer depends on O²⁻ ions diffusing inward, and the growth of the oxide film disposed on the outer layer depends on Fe²⁺ ions diffusing outward, thus a growth rate of the oxide film may be described by the parabolic rate law. Researches show that the 9% Cr martensite heat-resistant steel oxidation kinetic model is basically in accordance with a parabolic model.

Assuming that the growth of the oxide film/layer is controlled only by the inward diffusion of oxygen ions, the oxygen concentration at the metal/oxide layer interface is el. The oxygen concentration at the oxide layer/water vapor interface is c₀. The thickness of the oxide layer is X, accordingly a concentration gradient in the oxide layer is

$$\frac{d c}{d X}$$

and a diffusion coefficient of oxygen is D, the oxygen flux per unit time at the interface S is

$$d n = D \cdot S \frac{d c}{d X} d t$$

determined according to the Fick's first law.

Under a condition of steady-state diffusion,

$$\frac{d c}{d X} = \frac{{c}_{0} - {c}_{1}}{X}$$

is constant, the diffusion speed at an unit interface is

$$v = \frac{d n}{s d t} = D \frac{{c}_{0} - {c}_{1}}{X} .$$

Because the oxygen concentration at the interface of the metal/oxide layer is very low, the interface reaction is fast, and oxygen is not accumulated, therefore c₁ approaching 0 which is negligible; when the ambient oxygen concentration (oxygen partial pressure) is constant, c₀ may be regarded as a constant, the oxidation rate is inversely proportional to the oxide layer thickness. Similarly, the process of metallic cations diffusing outward may also be analyzed according to the above process, as long as the concentration difference of metal cations at the two interfaces is a constant value, the growth speed of the oxide layer is only inversely proportional to the thickness of the oxide layer:

$$v = \frac{d X}{d t} = D \frac{{c}_{0} + {c}_{0}^{′}}{X} = \frac{{k}_{p}}{X} \qquad ( 1 )$$

wherein c₀′ is the concentration difference of the metallic cations at the two interfaces.

Integrating the formula (1) to obtain X²=2kₚt, or is written as

$$X = {( 2 {k}_{p} )}^{\frac{1}{2}} {t}^{\frac{1}{2}} .$$

In the formula, kₚ is a constant related to the diffusion coefficient, generally abided by the Arrhenius equation:

$${k}_{p} = {k}_{0} exp ( \frac{- Q}{R T} ) ,$$

wherein Q is an activation energy, R is a gas constant, k₀ is a constant, kₚ is brought into the foregoing formula to obtain:

$$X = {( 2 {k}_{0} )}^{\frac{1}{2}} exp ( \frac{- Q}{2 R T} ) {t}^{\frac{1}{2}}$$

the constant coefficient is expressed by A, further it is simplified as follows:

$$X = A exp ( \frac{- Q}{R T} ) {t}^{\frac{1}{2}} \qquad ( 2 )$$

The experimental result shows that, an expression formula of the metal high-temperature oxidation kinetics generally is X=Atⁿ, which the index n in the formula is no need to be equal with ½ which is different from formula (2), but fluctuates within a certain range, such as a cubic law when n is ⅓. The research shows that the high-temperature steam oxidation model of the 9% Cr martensite heat-resistant steel also meets same regularity that X=Atⁿ. For different steel types and working conditions, values of the coefficient A and the index n are different. Therefore, the formula of the high-temperature steam oxidation film thickness of the 9% Cr martensite heat-resistant steel can be initially defined as follows:

$$X = A exp ( \frac{- Q}{R T} ) {t}^{n} \qquad ( 3 )$$

The present disclosure collects a large amount of practical experimental data that include the temperature of 550-700° C., the steam pressure of 23.0-35.0 MPa, the thickness data of the oxide film of the 9% Cr martensitic heat-resistant steel at the oxidation time of 200-20000 h, the parameters n, Q and A in equation (3) are calculated using the above data:

The calculation method is as follows:

step 1, solving n, and taking logarithm of two sides of the formula (3) to obtain:

$$ln X = ln A + ( \frac{- Q}{R T} ) + n l n t$$

letting the temperature T be a constant value, then

$$ln A + ( \frac{- Q}{R T} )$$

is a constant, denoted C, the above formula can be simplified to InX=C+nInt; Substituting the experimental data at various temperatures into the formula for linear fitting to obtain a fitting formula as follows:

when t=550° C.: ln X=0.017+0.501 ln t;

when t=600° C.: ln K=0.37+0.55 ln t;

when t=650° C.: ln X=3.14+0.255 ln t;

when t=700° C.: ln X=1.04+0.64 ln t.

It can be found that the value n is basically close to 0.5, which means the oxidation kinetics of the 9% Cr martensitic heat-resistant steel is basically in accordance with the parabolic law, so that if n is 0.5, the formula (3) is modified as follows:

$$X = A exp ( \frac{- Q}{R T} ) {t}^{0.5} \qquad ( 4 )$$

step 2, solving the activation energy Q, and taking logarithm of two sides of the formula (4) to obtain:

$$ln X = ln ( A {t}^{0.5} ) - \frac{Q}{R T}$$

letting t be a constant, then In (At^{0.5}) is recorded as a constant C, and the above formula is simplified into:

$$ln X = C - \frac{Q}{R T} \qquad ( 5 )$$

When t is 200h, the obtained experimental data is substituted into the fitting formula obtained from the step 1 to calculate ln X values at 550° C., 600° C., 650° C. and 700° C. respectively. Then substitute ln X to formula (5), Q is calculated to be 41628.07 when t is 200h. Similarly, Q can be calculated at other time as shown in Table 1, Q may be different at different time which indicates that the activation energy of the oxidation reaction is different at different time periods. The research shows that the oxidation reaction of 9% Cr is a complex and dynamically changing process; the reaction mechanism, generated products, and compositions and structures of the oxide materials are different at different stages of oxidation. Therefore, a mathematical model is used to fit the change of the activation energy along with the time to obtain an exponential model therein the activation energy Q and the time t are highly consistent, the obtained fitting formula is as follows:

Q=(39659.32+10.29)t⁽0.0092±3.52E4) (6)

and substituting the formula (6) back to the formula (4) to obtain a corrected thickness formula as follows:

$$X = A exp ( \frac{- 39659.32 {t}^{0.0092}}{R T} ) {t}^{0.5} \qquad ( 7 )$$

TABLE 1 shows different activation energy at different times

| t/h | Q/J · mol⁻¹ |
|---|---|
| 200 | 41628.07 |
| 600 | 42057.09 |
| 1000 | 42256.60 |
| 1500 | 42414.76 |
| 20000 | 43426.66 |

Step 3, solving a constant A, substituting the experimental data into the formula (7) to perform nonlinear surface fitting, to calculate A to be 11616.83, so that the obtained fitting formula is finally as follows:

$$X = 11616.83 exp ( \frac{- 39659.32 {t}^{0.0092}}{R T} ) {t}^{0.5} \qquad ( 8 )$$

In the above formula, unit of the temperature T is ° C., unit of time t is h, and unit of the thickness X of the oxide film is μm, the applicable time range of the formula is 200-20000 h, the temperature range is 550-700° C., and the steam pressure range is 23.0-35.0 MPa.

### Embodiment One

Comparison of the calculation methods involved in the present application with the experimental results of oxidation of T91.

In 2013, the oxidation situation of T91 steel under the conditions of 26 MPa, 600° C./650° C./700° C. is reported by Yunhai M A et al, the experimental conditions are respectively substituted into the formula provided by the instant application. The thickness of the oxide film calculated by the instant application is compared with the thickness obtained by experimental measurement, and the comparison results are shown in Table 2. It can be seen that the calculated thickness is very close to the thickness as experimentally measured.

TABLE 2
comparison of calculated thickness in the instant application
to the thickness in actual measurement.

| Tem- | | Measured | Calculated | | Error |
|---|---|---|---|---|---|
| perature/ | Time/ | thickness/ | thickness/ | Absolute | percentage/ |
| ° C. | h | μm | μm | error/μm | % |
| 600 | 1100 | 75 | 80.2 | 5.2 | 6.9 |
| 650 | 500 | 106 | 109.8 | 3.8 | 3.6 |
| 700 | 1000 | 238 | 258.2 | 20.2 | 8.5 |

### Embodiment Two

The calculation method provided by the instant application is compared with the experimental result of T/P92.

The oxidation experiment of P92 steel at 550° C. and 25 MPa, for 600h was reported by Zhongliang Zhu et al in 2013, and the thickness of the oxide film measured from the sectional SEM image was about 28 μm. By substituting the experimental conditions into the fitting formula of the instant application, the thickness of the oxide film obtained is calculated to be 28.8 μm, which is very close to the measurement result, and the error percentage is only 2.8%.

### Embodiment Three

The calculation method in the instant application is applied in a real power plant environment.

Data of the steam oxide-scale thickness in the tubeline operated in the power plant which is recorded in a design guidance for preventing steam oxidation, gas corrosion and erosion on the heated surface of the pulverized coal boiler of the large power station shows that, under a condition of 600° C. and 25 MPa, the thickness of the oxidation scale of the T92 tube is 376 μm after 22981 h of operation. The above parameters are substituted into the formula of the instant application to obtain the fitting formula, the thickness of the oxide film is calculated to be 367.14 μm, and the deviation is only 2.4 percent compared with a measurement result, which means that the fitting formula performs well in practical application.

### Embodiment Four

The calculation method of the instant application is applied in an actual power plant environment.

The steam pressure of an ultra-supercritical unit used in a certain power plant at abroad is about 28.4 MPa. Under a condition that a superheater tube is made of T92 material and operates for about 15000 hours at the temperature of 600° C., the thickness of the oxide scale in the tube is measured to be about 215 μm. And the thickness of the oxide scale is calculated to be about 241 μm by substituting operating parameters into the formula as disclosed in the instant application, with a deviation of 26 μm and an error percentage of 12.1%.

### Embodiment Five

The calculation method as disclosed in the instant application is applied in the actual power plant environment.

The tube near outlet area of a high-temperature superheater of a 600 MW supercritical once-through boiler is made of a T91 material in a certain domestic power generation company, the steam temperature near outlet area is about 580° C., the steam pressure is about 26 MPa, and the thickness of oxide scale in the tube is about 214 μm after running for 20000 hours. Substituting above operating parameters into the formula as disclosed in the instant application, the thickness of the oxide scale is about 201.5 μm, and the error percentage is 5.8%.

The above embodiments all show that the thickness of the oxide scale/film/layer of the 9% Cr martensitic steel calculated by the aforementioned method is in good accordance with the actual measurement result, and the error percentage is controlled within 15%.

The technical solutions of the instant application are not limited to the above embodiments, and all technical solutions obtained by using equivalent substitution methods fall within the scope of the instant application.

What is claimed is:

1. A method for calculating a thickness of an oxide film of a martensite heat-resistant steel under supercritical high-temperature steam, comprising:
the martensite heat-resistant steel is a 9% Cr martensite heat-resistant steel; and
$$X = A exp ( \frac{- Q}{R T} ) {t}^{n}$$
a formula for calculating the thickness of the oxide film is
wherein X is the thickness of the oxide film (μm), A is a constant coefficient, Q is an activation energy (J·mol⁻¹), R is a gas constant, T is temperature (° C.), and t is time (h).

2. The method according to claim 1, wherein the method comprises: the temperature of the high-temperature steam T is in a range of 550-700° C., the steam pressure is in a range of 23.0-35.0 MPa, and the time t is in a range of 200-20000 h.

3. The method according to claim 1, wherein n is 0.5.

4. The method according to claim 1, wherein a mathematical relationship between the activation energy Q and the time t is: Q=39659.32t^{0.0092}.

5. The method according to claim 1, wherein the constant coefficient is 11616.83.

6. The method according to claim 1, wherein Q is 41628.07 when oxidation time is 200 h.

7. The method according to claim 1, wherein Q is 42057.09 when oxidation time is 600 h.

8. The method according to claim 1, wherein Q is 42256.60 when oxidation time is 1000 h.

9. The method according to claim 1, wherein Q is 42414.76 when oxidation time is 1500 h.

10. The method according to claim 1, wherein Q is 43426.66 when oxidation time is 2000 h.
